(19) United States
(12) Patent Application Publication
(10) Pub. No.: US 2007/0128612 A1
(43) Pub. Date: Jun. 7, 2007

(54) DNA family tree kit

(76) Inventor: Barbara J. Povlich, West Allis, WI (US)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 11/294,127

(22) Filed: Dec. 5, 2005

(51) Int. Cl. C12Q 1/68

(52) U.S. Cl. 435/6

(57) ABSTRACT

A DNA family tree kit includes a storage case configured to receive a plurality of DNA storage boxes, a variety of mechanisms for collecting samples of DNA family members and a genealogical organization system that includes a family tree. A method of collecting, storing and categorizing DNA includes collecting at least one sample of DNA from a family member, storing the sample in a DNA storage box, placing the box in a storage case and recording information about the family member in a genealogical organization system that includes a family tree.

### BACKGROUND OF THE INVENTION

1. Field of the Invention

The present invention relates in general to the field of DNA collection and storage. More specifically, the invention relates to a kit designed for home use that allows DNA samples and personal information from multiple members of a family to be collected, categorized and maintained for generations in a manner that illustrates the genetic predisposition to certain illnesses of the family members.

2. Discussion of the Related Art

The occurrence of genetic disease within humans remains to be completely understood. However, in recent years it has become apparent that many common diseases are directly related to a human's genetic makeup. It is well established that genetic and environmental factors can affect whether a human develops and suffers the symptoms of a particular disease. The weight of each factor often varies from disease to disease. In many cases, an individual may carry the genetic code for a disease but never develop the disease or show symptomatic manifestations. However, such individuals may pass the genetic code to offspring, and thereby pass down the potential for developing the disease to future generations. Much of the recent study of human genetics has been connected with the study of diseases. As more genetic research is performed, it has become evident that many common diseases are in fact genetic diseases.

The unit of heredity is the gene, which is part of a complex protein molecule called deoxyribonucleic acid (DNA). Popularized in recent years by its use in high-profile criminal investigations and paternity cases, DNA is most commonly used to prove relationships between individuals. New tests have also turned DNA into a popular tool for determining ancestry and the probability of one having a disease.

Chromosomes are made up primarily of DNA and can be thought of as being composed of a linear series of genes each containing instructions for producing one specific body trait. Thus, a chromosome is like a string of beads, each bead being a gene that influences some physical characteristic. The location of the gene on the chromosome is called its locus. The gene at any locus can normally exist in one of several forms.

Gene transmission involves two pairs of genes, one pair from each parent. In reproduction, these pairs split, and the child receives one member of each pair, chosen at random from each of his/her parents. Humans, for instance, have 46 chromosomes, 23 of which are acquired from the father and are matched by 23 from the mother. Therefore, each human carries part of the genetic information that determines the genetic make up of his/her offspring.

There are numerous examples of diseases known to have a genetic component. Such examples include Alzheimer's, cystic fibrosis, Huntington's, and bipolar disorder. Each of these diseases will be discussed briefly in turn.

Nearly 4 million Americans have Alzheimer's disease, which is a progressive dementia that robs people of their memories. With people living longer and the aging of the “Baby Boom” generation, the population afflicted with the disease is rapidly rising. About 20 million Americans are expected to be diagnosed with Alzheimer's by the year 2050. Although there is still no cure for Alzheimer's, two decades of research have yielded new drugs and treatments that are providing some optimism for Alzheimer's patients and their caregivers. New research not only emphasizes treatment, but also prevention and early recognition of the disease. Evidence shows that the earlier Alzheimer's patients are treated with the available drugs, the better the results. One key to identifying Alzheimer's is the identification of an individual's genetic disposition to the disease.

Cystic fibrosis is a common genetic disease that kills children. The disease results from a disorder in the genes of its victims that cause cells to produce a thick mucous secretion which clogs the passageways of many vital organs. A child infected with cystic fibrosis will most likely not live to be an adult. Cystic fibrosis is caused by a single abnormal allele. The disease will occur only in children who have two copies of this abnormal allele, one from each parent. Such diseases are said to be recessive diseases. If two healthy parents have a child afflicted with cystic fibrosis, then both parents are carriers of the abnormal allele. The probability that two healthy carriers of the abnormal allele will produce a child with cystic fibrous is ¼. The probability of two carriers producing a carrier is ½.

Huntington's disease is a disease caused by a disorder of the genes, which causes its victims to have jerky, involuntary motions and extreme mental changes. The disease appears in adults after they have reached middle age. By this time, many of the victims have had children already, and the concern about whether the parent has passed the disease to his or her offspring becomes intense. As with cystic fibrous, Huntington's disease is caused by a single abnormal allele. However, Huntington's disease occurs in people who have only one copy of the abnormal allele. Thus, if either parent passes on this abnormal allele, the child will be inflicted with the disease. Such diseases are said to be dominant diseases. Huntington's is a very rare disease. Its victims are almost always married to people with two normal alleles. In the case of Huntington's, if one parent has a normal genotype and other is afflicted, the chances are ½ that the child will eventually develop the disease.

Bipolar disorder is an illness of moods, in which a person has mood swings that are greatly exaggerated, from severe depressions to wildly high manias. It is estimated that 1 to 5 percent of the population is afflicted with bipolar disorder. In population studies, they found that there is a 10 percent risk that others in the nuclear family (father, mother, siblings) will have the disorder once one family member is diagnosed. Second degree relatives, such as grandparents, uncles, and aunts were found to have a four percent risk. Bipolar disorder tends to run in families and is believed to be inherited in many cases. Effective treatments are available that greatly alleviate the suffering caused by bipolar disorder and can usually prevent its devastating complications. Like other mental illnesses, bipolar disorder is also hard on spouses, family members, friends, and employers. Family members of people with bipolar disorder often have to cope with serious behavioral problems and the lasting consequences of these behaviors. These include marital break-ups, job loss, alcohol and drug abuse, and suicide. An early diagnosis of bipolar disorder may decrease the chance that a patient and patient's family suffer such consequences.

In addition to the above listed examples, numerous cancers were found to have a genetic component to them. Some cancers that appear to be influenced by genes (estimated percentage of genetic contribution also shown) include; prostate cancer—42%; colorectal cancers—35%; stomach cancer—28%; breast cancer—27%; and lung cancer—26%. In addition, a small minority of cancers (family cancer syndromes) are clearly inherited, e.g. familial polyposis coli, familial non-polyposis colon cancer, some cancers of the breast and some thyroid cancers. Familial cancers are usually due to inherited mutations in a single growth-controlling gene and show a dominant, late-onset inheritance pattern. In common conditions such as coronary heart disease or cancer of the colon or breast, it can be difficult to distinguish familial from sporadic cases.

Other diseases that are thought to have an important genetic component including coronary heart disease, hypertension, diabetes, psoriasis, rheumatic disease, thyroid disease and schizophrenia. Most of these groups of disorders include some cases caused by genetic factors alone, some caused by environmental factors alone and some related to an interaction. When genetic studies, particularly DNA analysis, succeed in unraveling the genetic element in such disorders, it often emerges as more important than has been anticipated. For example, genetic factors are now known to be particularly important in non-insulin-dependent diabetes mellitus.

The above examples are only a few of the many diseases and disorders currently known to have a genetic component to them. It is well established that family disease studies provide important information relating to the likelihood that an individual will suffer from a genetic disease. Such studies speak to some kind of gene, or exposure, or custom, or practice that figures in the causes of workings of an illness.

In addition to the value in predicting the onset of a genetic disease, genetic analysis can also help determine an effective treatment for both genetic and non-genetic diseases. Effective and safe treatment of disease varies from patient to patient. A course of treatment can vary from restoring wellness to causing death. According to statistics, more than 100,000 people die each year from adverse responses to medications. Another 2.2 million experience serious reactions, while others fail to respond at all. DNA variants in genes involved in drug metabolism encode enzymes. Enzyme function affects patient response to both the drug and the dose. Identifying and matching such DNA variants among family members having and being treated for the same or similar diseases provides direction in prescribing the most effective medications and dosages.

From the above examples it is evident that there is a value in maintaining and studying the genetic make-up of individuals throughout their ancestry. In addition to the value in understanding the familial genetic makeup across lineages for the purpose of identifying and treating diseases, there also remains a value in maintaining samples of ones DNA in the event that someone is the victim of a violent crime. Hair sample comparison and DNA print matching, for example, might be used by forensic pathologists to identify an unknown hair, blood, or tissue sample as that of a missing person, thereby generating a lead to locating such person. Tragically, some missing persons are victimized and killed by their abductors who often leave their victim's body in an isolated or inaccessible location. When such bodies are eventually found, they are often so deteriorated that identification through traditional means is impossible. In these instances, techniques such as DNA print matching can still provide a positive identification since every cell in an individual's body, even the cells of deteriorated remains, contains DNA cells having gene patterns that are unique to that individual alone.

In addition to the value in maintaining the DNA of humans, there are additionally several important applications for a DNA collection and storage mechanism for plants and animals. Well organized genetic information would be very important to animal breeders and plant nurseries as well in order to develop disease resistant crops or a better breed of animal.

The broad concept of DNA banking or storage is generally known in the art. Many techniques are used to collect samples of an individuals DNA. For example, a body sample such as blood or cheek cells or other body tissue may be collected. Such samples, however, are often stored in a large commercial place of business, a research laboratory or a university. Such institutions may use complex procedures and preservation conditions. In addition, commercial institutions which offer banking of DNA and other genetic material might decide to discontinue the offering of this service, go out of business, be the target of a business takeover, or change its array of services provided. If these events happen, it leaves the owner of the DNA/genetic samples with the possibility of their samples being lost, transferred to a new place of storage, etc.

There is also a previously unrecognized advantage to collecting multiple samples of DNA from a variety of sources on an individual. Most individuals, companies, institutions rely on the collection and banking of one sample of genetic material for possible future use. The collection of genetic material from several different human body sites for possible future use in genetic analysis or genetic testing represents an improvement over the collection of samples from one body site. There are different types of genetic mutations namely germ line and somatic cell. It is likely that a germ line mutation would probably be present in most if not all of the cells of an individual's body. In this case, one DNA gene sample would probably give a fairly accurate account when that DNA was analyzed and tested for the genetic aberrance. A germ line mutation occurs or is present in the egg or sperm which together form the zygote at the beginning of development. This is why most if not all of the cells would carry the genetic alteration being sought. However, the somatic cell mutation or genetic alteration may happen during or after development. Thus, only cells that originate or come from that cell would carry the mutation and the body would show mosaicism. It may be then that a sample taken from one body site might not have come from the line of cells produced from the mutated somatic cell. In that event, the genetic alteration being sought would be missed if only that sample was available for testing. Thus it is advantageous to have more than one genetic DNA sample available for testing.

For example a preferred system would allow samples to be collected from the mouth cheek cells which are derived from ectoderm, hair follicles which are derived from ectoderm and mesoderm, and blood which is derived from mesoderm. In order to collect material from the endoderm, the sample may be taken from urethral discharge, urine, urine sediment, urine filtrate (urine passed through a filter and the filtered material collected and stored) which may contain endoderm and/or mesoderm. Another possibility is to collect and store some type of material from the lining of the digestive tract which is derived from endoderm. Feces could possibly be dried, filtered, centrifuged, or processed in some type of fashion suitable for DNA collection and storage/preservation. Another possibility is the collection of some type of respiratory tract material or secretion which contains endodermal material.

Some attempts have been made to provide a DNA home storage mechanism. Such prior art devices, however, often include complex mechanisms, limited collection resources and additionally do not provide a good means of organization. For example, U.S. Pat. No. 5,101,970 to Turner, the disclosure of which is hereby incorporated by reference, discloses a system for collecting and storing DNA specimens from living persons which includes storage of the specimens together with information in a freezer. However, the information is collected only from living parties and blood samples are used. The disclosure is further limited to storage in a freezer. There is no comprehensive organization or display method.

U.S. Pat. No. 5,856,102 to Bierke-Nelson et al., the disclosure of which is hereby incorporated by reference, discloses a process and method of DNA banking in which DNA or other genetic material is collected, stored, preserved and banked in a home/self-storage setting. The invention includes kits designed to collect and bank DNA and other genetic material in a home/self-storage setting. The objective of the invention is to preserve genetic material in the event that it is needed for genetic analysis, genetic testing, genetic diagnosis, genetic therapy, forensic analysis, or identification. The kit does not provide for the organization of multiple samples in a manner that illustrates familial relationships, and genetic predispositions across generations.

U.S. Pat. No. 6,291,171 to Riccardi et al., incorporated by reference herein, discloses a kit for collecting and transporting DNA specimens from an individual for analysis or study includes swabs for harvesting DNA cells from the mouths of individuals and corpses. The kit consists of a rectangular panel having a center portion and two hinged members. The center portion is provided with a folded stand with apertures for holding swabs containing the DNA specimens. The kit also does not provide for the organization of multiple samples in a manner that illustrates familial relationships and genetic predispositions across generations.

What is therefore needed in light of the above is a system of collecting, preserving and organizing genetic information about humans and other organisms. Such a system should be complete, convenient, easy to use and store in the home without medical supervision and should be adapted to preserve cell bearing specimens for long periods without significant deterioration of the specimens. The system should preferably be self contained and include a mechanism for easily organizing and displaying the genealogical relationship between family members as well as their predisposition to genetic diseases. The system should also have the capability to gather cells from each of the three embryonic germ layers of cells: ectoderm, mesoderm, and endoderm.

### SUMMARY AND OBJECTS OF THE INVENTION

In view of the foregoing, it is one object of the present invention to provide a DNA family tree kit for collecting, preserving and organizing genetic information about humans and other organisms. It is a further object of the invention to provide a kit that is convenient, easy to use and store in the home without medical supervision. It is another object of the present invention to provide a kit that is self contained and includes a mechanism for easily organizing and displaying the genetic genealogical relationship between family members as well as their predisposition to genetic diseases. It is another object of the present invention to provide a kit that has the capability to gather cells from each of the three embryonic germ layers of cells: ectoderm, mesoderm, and endoderm.

Consistent with the foregoing objects and in accordance with the invention as embodied and broadly described herein, a method for collecting, storing and categorizing DNA and other genetic material across generations of a family, a DNA family tree kit and a method of assimilating and preserving genetic information about a family over multiple generations are disclosed in suitable detail to enable one of ordinary skill in the art to make and use the invention.

In one embodiment, a method for collecting, storing and categorizing DNA and other genetic material across generations of a family includes the steps of collecting at least one sample of DNA from a family member, storing the sample in a DNA storage box, placing the box in a storage case and recording information about the family member in a genealogical organization system that includes a family tree having indicia indicating the location of the family member's DNA storage box in the storage case.

The step of collecting DNA may include the step of collecting a blood sample. The blood sample may be collected by placing drops of blood on a card labeled for identification, drying the blood and placing the card in a plastic sleeve. In another embodiment, the step of collecting DNA may include collecting cheek cells. The step of collecting cheek cells may be performed brushing the inside of the mouth cheek to collect cells with a swab, drying the swab and placing the swab in a sleeve with a card labeled for identification. In yet another embodiment, the step of collecting DNA comprises collecting a hair follicle. The collection of the hair follicle could be performed by collecting loose head hair which accumulates in a comb, cutting off and discarding the end of the hair not containing hair follicles and placing the hair strands containing hair follicles in a sleeve with a card labeled for identification. In one embodiment, the method includes the step of taking fingerprints of the family member and placing the fingerprints in the DNA storage box. In another embodiment, the step of recording information about the family member in the genealogical organization system includes the step of filling out an information packet on an individual including information on an individual's name, date of birth, date of death, sex, mothers maiden name, country of origin, eye color, hair color, vision, blood type, height, weight, diseases and age of incidents, injuries and age of incidents and prescriptions taken.

In another embodiment, the family tree includes indicia indicating the location of the information packet in the storage case, whether a person is a known carrier of a genetic disease and indicating a person's response to medication.

In still another embodiment, a DNA family tree kit includes a storage case configured to receive a plurality of DNA storage boxes, a means for collecting at least one sample of DNA from a family member and a genealogical organization system that includes a family tree. The family tree may include indicia indicating the location of a family member's DNA storage box within the storage case, if a family member is a known carrier of a genetic disease and a family member's response to medication. The kit may also include an information packet in the storage case.

In a final embodiment, a method of assimilating and preserving genetic information about a family over multiple generations includes the steps of collecting data about the family members, collecting at least one sample containing DNA from each of the family members, sealing the collected samples against exposure to ambiance, storing each family member's DNA sample in a separate DNA storage box, placing the DNA storage boxes in a storage case and recording the data about the family member in a genealogical organization system that includes a family tree having indicia indicating if a family member is a known carrier of a genetic disease.

These, and other, aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

A clear conception of the advantages and features constituting the present invention, and of the construction and operation of typical mechanisms provided with the present invention, will become more readily apparent by referring to the exemplary, and therefore non-limiting, embodiments illustrated in the drawings accompanying and forming a part of this specification, wherein like reference numerals designate the same elements in the several views, and in which:

FIG. 1 is a perspective view of a closed DNA family tree kit constructed according to the present invention;

FIG. 2 is a perspective view of an opened DNA family tree kit with a removable and reversible article holding board exploded therefrom;

FIG. 3 is a perspective view of one side of the article holding board of FIG. 2;

FIG. 4 is a perspective view of the other side of the article holding board of FIG. 2;

FIG. 5 is a schematic view of the components of the DNA extraction kit of the DNA family tree kit;

FIG. 6 is a schematic view of the family tree of the DNA family tree kit;

FIG. 7 is a first page of an information packet of the present invention; and

FIG. 8 is a second page of an information packet of the present invention.

In describing the preferred embodiments of the invention that are illustrated in the drawings, specific terminology will be resorted to for the sake of clarity. However, it is not intended that the invention be limited to the specific terms so selected and it is to be understood that each specific term includes all technical equivalents that operate in a similar manner to accomplish a similar purpose. For example, the word “connected” or terms similar thereto are often used. They are not limited to direct connection but include connection through other elements where such connection is recognized as being equivalent by those skilled in the art.

### DESCRIPTION OF PREFERRED EMBODIMENTS

The present invention and the various features and advantageous details thereof are explained more fully with reference to the non-limiting embodiments described in detail in the following description.

1. System Overview

The present invention relates to a DNA family tree kit and method of storing DNA that includes an organization system that displays about the genealogical relationship between family members as well as their predisposition to genetic diseases. The kit includes a storage case configured to receive a plurality of DNA storage boxes a variety of mechanisms for DNA from family members and a genealogical organization system that includes a family tree.

2. Detailed Description of the Preferred Embodiments

Specific embodiments of the present invention will now be further described by the following, non-limiting examples which will serve to illustrate various features of significance. The examples are intended merely to facilitate an understanding of ways in which the present invention may be practiced and to further enable those of skill in the art to practice the present invention. Accordingly, the examples should not be construed as limiting the scope of the present invention.

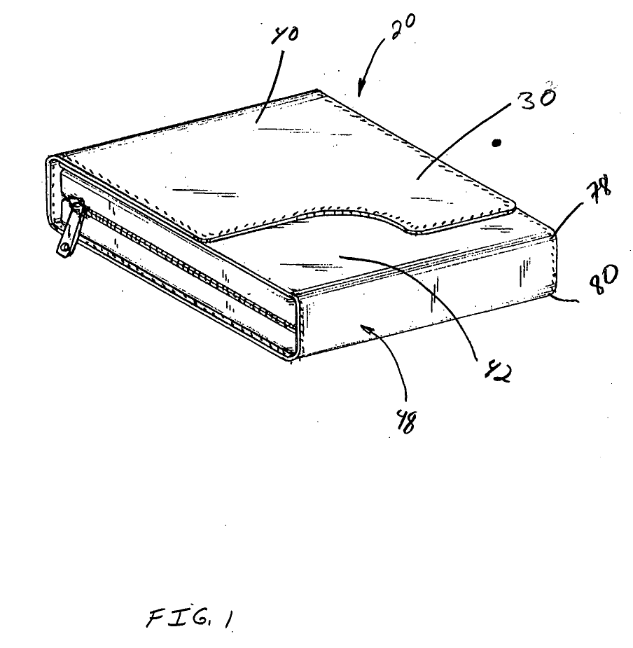

Referring now in more detail to the drawings, in which reference numerals refer to salient elements of the preferred embodiment, FIG. 1 illustrates a preferred embodiment of the DNA home storage kit 20 that embodies principles of the invention in a preferred form. The kit 20 includes a storage case 30, a multi-source DNA extraction kit 24, and a genealogical organization system 26 housed within the storage case 30.

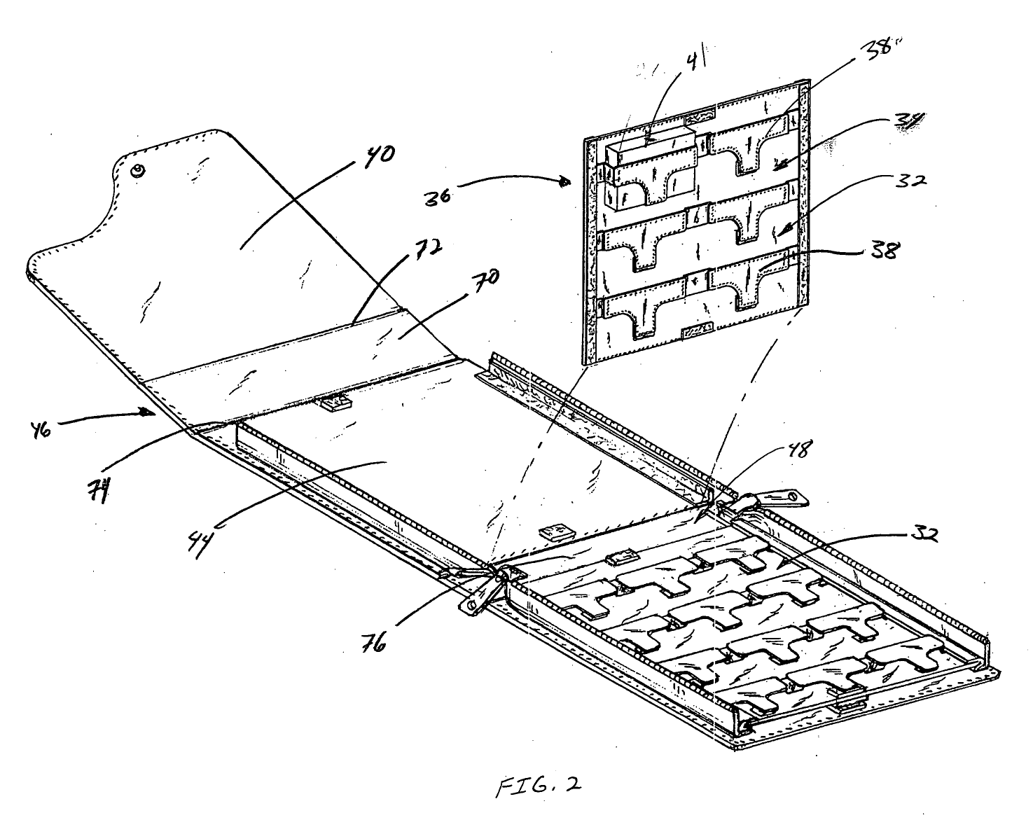

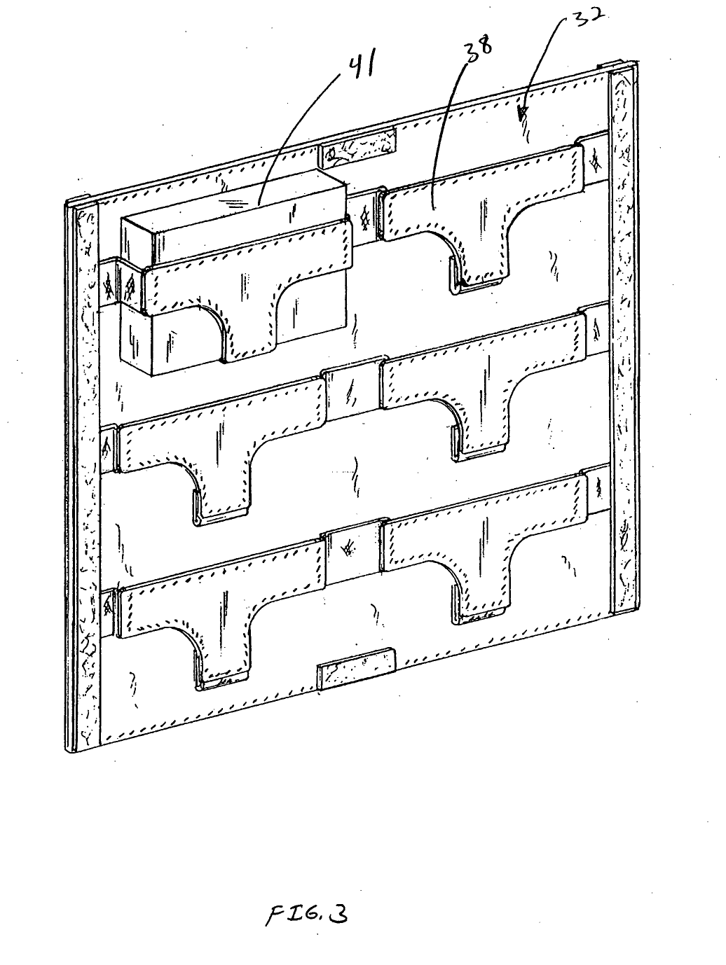

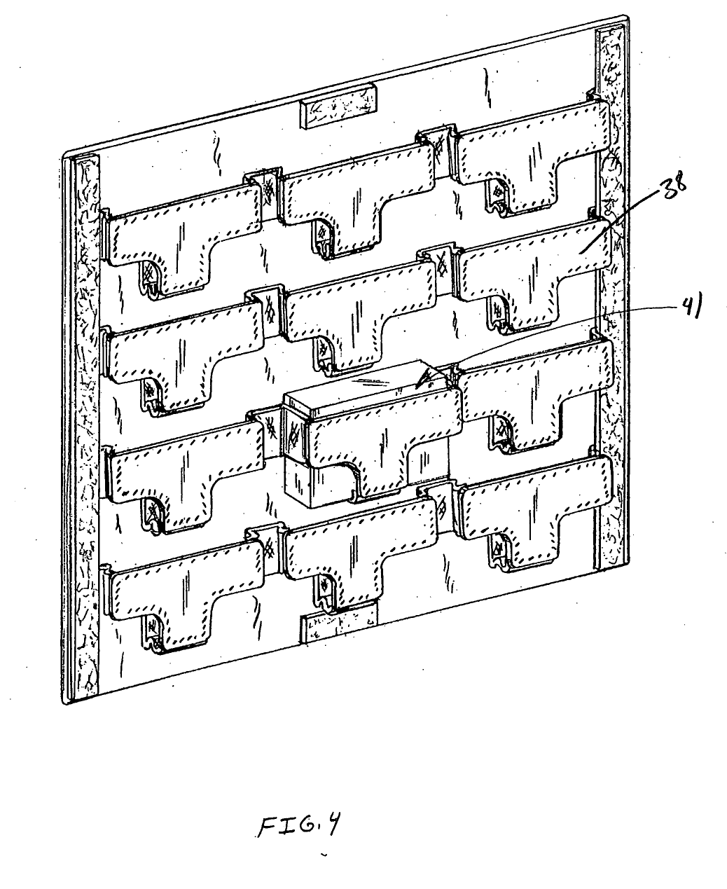

FIGS. 1-4 illustrate one preferred embodiment of the storage case 30, which is equipped with a plurality of article holding boards 32, each of which is removable and reversible. It should be understood that a wide variety of alternative storage cases could be utilized with the present invention and the disclosed storage case should not be considered limiting. Each article holding board 32 has a front surface 34 and a back surface 36 that each carries a plurality of storage containing pockets 38. Although not shown, the case 30 may include a binder within having at least one two or three ring binder assembly for retaining the holding boards 32, DNA extraction kit 24, and components of the genealogical organization system 26.

The case 30 has a plurality of cover panels with one of the cover panels foldable relative to another one of the cover panels. In the preferred embodiment shown in FIGS. 1-4, the portfolio 30 has three cover panels 40, 42, and 44 with two of the cover panels 40 and 42 being outer cover panels. A first one of the outer cover panels 40 is attached by a first hinge arrangement 46 to a middle cover panel 44 and a second one of the outer cover panels 42 is attached by a second hinge arrangement 48 to the middle cover panel 44.

The first hinge arrangement 46 preferably includes a first spine 70 that is attached to outer cover panel 40 by a first hinge 72 and that is attached by a second hinge 74 to the middle cover panel 44. As is shown more clearly in FIG. 1, the second hinge arrangement 48 preferably includes a second spine 76 that is attached to outer cover panel 42 by a third hinge 78 and that is attached by a fourth hinge 80 to the middle cover panel 44.

One of the article holding boards 32 is removably and reversibly attached to outer cover panel 42 and the other one of the article holding boards 32 is removably and reversibly attached to middle cover panel 44. Each article holding board 32 can be oriented with its front surface 34 toward a user who has opened the case 30 to present a plurality of pairs of storage containing pockets 38 so they, along with any pocketed article, are each accessible to the user. Each article holding board 32 can be reversed or flipped around such that its rear surface 36 is disposed toward the user to present a plurality of pairs of storage containing pockets 38 so they, along with any pocketed article, are each accessible to the user. This can be done even if the pockets 38 on both surfaces 34 and 36 each contain an article. In the preferred embodiment, the article comprises a plurality of individual rectangular DNA storage containers or boxes 41 configured to retain samples and other personal information from individuals in a family as described in greater detail below. The storage boxes 41 are preferably sequentially numbered or include some other identifying indicia.

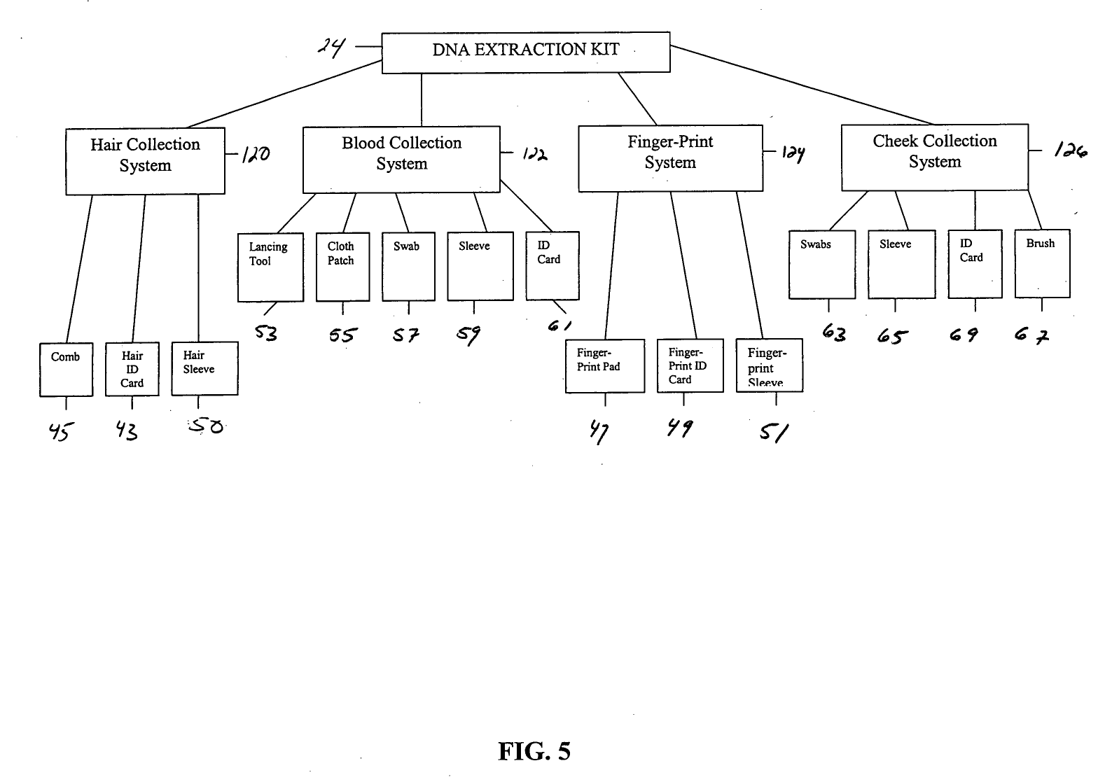

Turning to FIG. 5, housed with the storage case 30 of the kit 20 is a multi-source DNA extraction kit 24 provided with a number of instruments for collecting and preserving DNA from a specimen for identification storage and analysis. More specifically, the multi-source DNA extraction kit 24 includes a hair collection system 120, a blood collection system 122, a fingerprint collection system 124, and a cheek collection system 126.

The hair collection system includes a sealable plastic sleeve 50 for collecting and storing a sample of an individual's hair. The sleeve 50 includes an information card 43 bearing the date of the sample and the identity of the person from whom the sample was taken. Hair from the human head may collected using an ordinary comb 45 included with the kit. Loose hair which accumulates in the comb is taken. The ends are cut off and discarded and the remaining hair strands containing the hair follicles are placed in the sleeve 50. The sleeve 41 and identification card are then placed for storage in a DNA storage box 41.

The multi-source DNA extraction kit 24 also includes a fingerprint collection system 124. A disposable fingerprint pad 47 is impregnated with fingerprint ink and secured to a fingerprint identification card 49. An identification card 49 has printed on one side a matrix for receiving the fingerprints of an individual in the usual way. The array includes positions for fingerprints of each finger of each hand as well as positions for prints of several fingers taken in conjunction. Space for providing the identity of the person whose fingerprints appear on the card is also provided. Once the fingerprints have been collected, the pad 47 may be disposed of and the identification card 49 stored in a plastic sleeve 51. The sleeve 51 is then placed for storage in the DNA storage box 41.

The DNA extraction kit 24 also includes a blood collection system 122 for securing a sample of an individual's blood for use in DNA print identification. The blood collection system includes a lancing tool 53 for piercing the tip of a finger to draw blood, a cloth patch 55 for receiving and securing a sample of the blood, and a sterile swab 57 for wiping the pricked finger before and after the blood sample has been taken. A sealable plastic sleeve 59 is sized to receive and seal the patch 55 and blood sample thereon and an identification card 61 is provided for identifying the date of the sample and the person whose blood is contained within the patch 55. An individual may also have blood drawn by a licensed health care provider. In such cases the individual should request that drops of blood be applied to specialized identification card 61. The blood drops are allowed to dry and then the card 61 is placed in the sleeve 59 and the sleeve is placed in the DNA storage box 41.

The multi-source DNA extraction kit 24 also preferably includes a cheek collection system 126. The cheek collection system 126 includes swabs 63 for oral extraction that are preferably constructed from high modulus fibers such as Dacron®. The swabs 63 can successfully remove scrapings from the inner cheek. Where the sampled individual is a corpse, the swab heads 63 can comprise PCR or any other substance for fixing the DNA on the swab head. PCR (Polymerase Chain Reaction) technology can amplify a genetic blueprint a million fold as tiny segments of the human genomic DNA. Once the swabs 63 have dried, they are preferably stored in a swab sleeve 65 with an identification card 69. The drying of the swabs 63 is important to prevent the growth of fungus. Thus, the envelopes containing the swabs 63 are preferably water resistant. The card 69 is placed in the sleeve 65 and the sleeve 65 is placed in the DNA storage box 41.

Alternatively, a small brush 67 approximately 6 inches in total length with a handle approximately 5 inches in length and a set of bristles approximately 1 inch in length can be used to collect mouth cheek cells and scrapings. Following collection using specified instructions, the brush 67 is placed into the sleeve 65.

It is possible that DNA may be collected from other sources and stored in the DNA storage box 41. Samples may be collected from skin scrapings, nasal wash or scrapings or secretions, eye secretions, mucous membranes, saliva, expectorant, toenails, fingernails, sputum, urine, urine sediment, genital secretions, sperm, semen, vaginal and cervical secretions, feces, fecal matter or other body sites.

It should be recognized that the components of the DNA extraction kit 24 are preferably stored within the storage case 30. The extraction kit 24 may be housed within a pocket or held in a large sleeve or folder retained by a two or three ring binder of the storage case 30. The DNA extraction kit preferably includes a booklet that includes detailed instructions for use of the extraction kit 24. A set of decals each imprinted with the word “SEALED” may be provided for securing the collected data and specimens as detailed above. Bar codes or other indicia may be used to label the specimens or the boxes housing all of the specimens for an individual

All of the sleeves containing samples of DNA from the same individual are preferably stored in a single DNA storage box 41. The DNA is not extracted at the time of storage but can be extracted latter when and if a sample is to be tested and genetically analyzed. Once the samples have been collected and stored in the DNA storage box 41, the box 41 may be placed in an appropriate storage containing pocket 38 in the storage case 30.

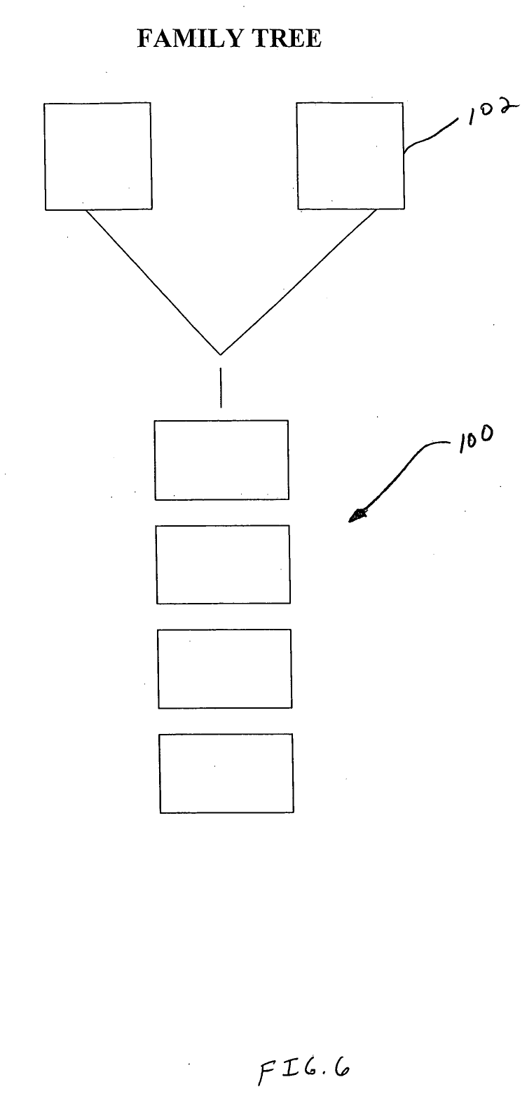

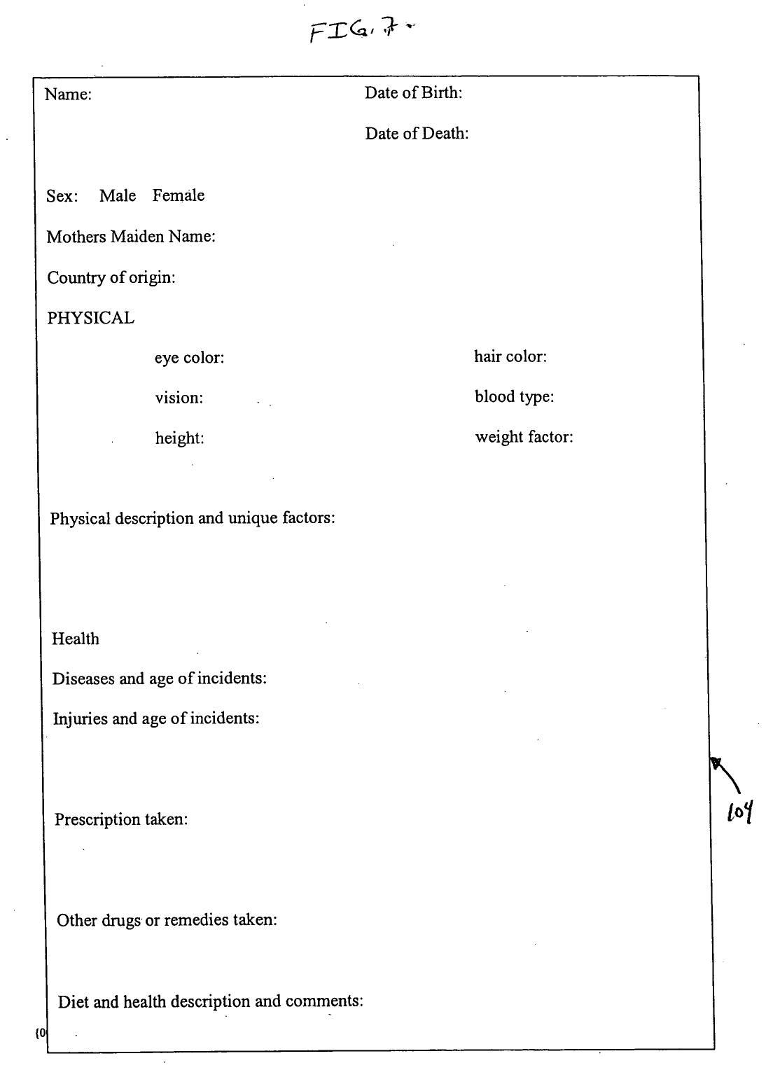

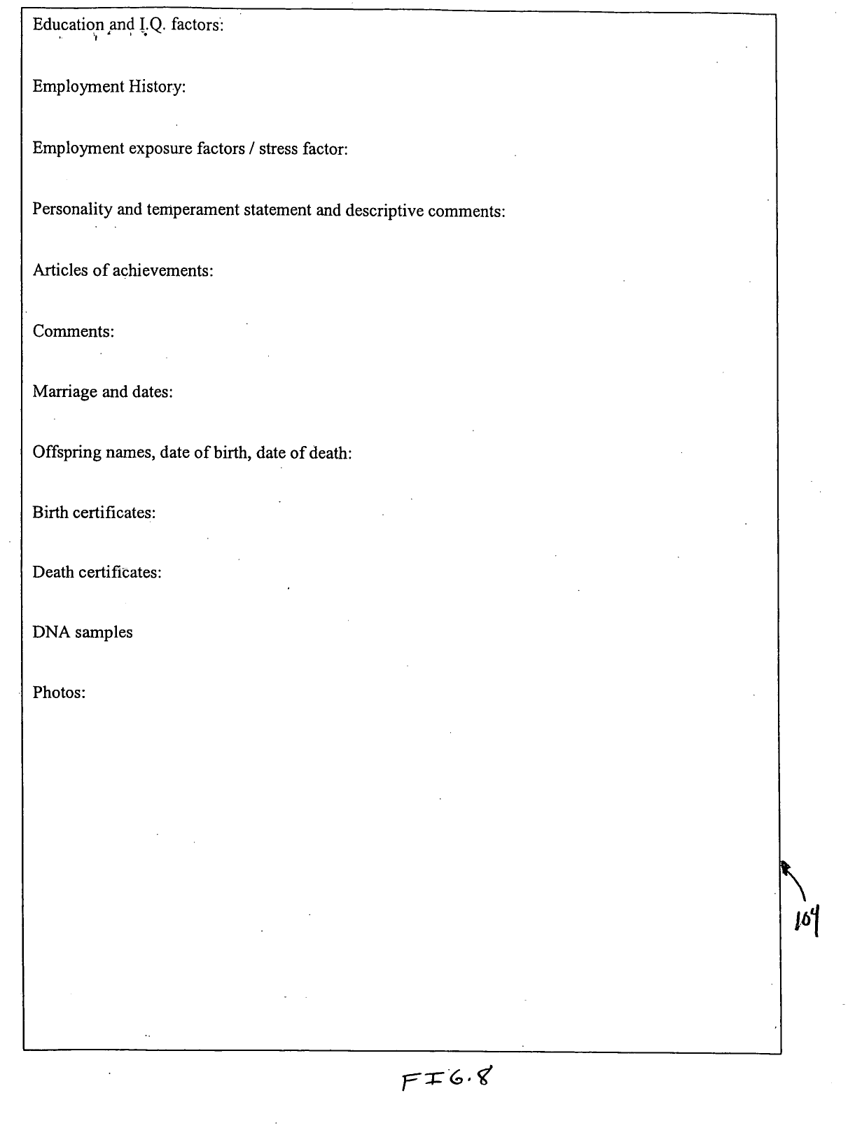

In addition to the DNA extraction kit 24 the kit 20 also includes a genealogical organization system 26 housed within the storage case 30. The genealogical organization system 26 includes a family tree 100 (FIG. 6) and multiple information packets 104 (FIGS. 7 and 8) not previously known in home storage kits or DNA collection systems. The genealogical organization system 26 allows a user to record, organize and easily view ones genealogy and genetic predisposition to several genetic diseases.

The genealogical organization system 26 preferably uses a simple numbering system that coordinates an individual's samples stored in the kit 20 with their position on a family tree 100. The components of the genealogical organization system 26 may be retained within the case 30, using a three ring binder or other known retaining feature. As illustrated in FIG. 6, the genealogical organization system 26 includes a family tree 100. The family tree 100 includes a number of blocks 102 for each individual of the extended family. Preferably, the blocks 102 include basic information about an individual such as the name of the individual, and the location/number of their corresponding storage box 41, as well as the location/number of their corresponding information packet 104.

In the preferred genealogical organization system 26, there is also some symbol or indicia on the block 102 of the family tree 100 indicating if the individual is/was a known carrier or suffered from a genetic disease. For example if an individual was a carrier for cystic fibrosis their block would include their name, storage box number, information packet number and a “CF Carrier” indicia indicating that the individual was a carrier for cystic fibrosis. Likewise, adverse responses to medications are also indicated on the block 102. For example, and adverse response to penicillin might be indicated “AD Penicillin.”

For each individual included in the family tree 100, there is preferably included an information packet 104 retained within the case 30. Each information packet 104 may be retained in the case 30 using a three ring binder or other known retaining feature. The information packet 104 is preferably numbered and the number is located in the individual's corresponding block 102 for ease of reference. The information packet 104 preferably includes sections for the inclusion of personal information and materials on the individual including: name; date of birth; date of death; sex; mothers maiden name; country of origin; eye color; hair color; vision; blood type; height; weight; a general physical description and listing of any unique factors; a general description of health; diseases and age of incidents; injuries and age of incidents; prescriptions taken; other drugs or remedies taken; diet and health description and comments; education and I.Q. factors; employment history; employment exposure factors/stress factor; personality and temperament statement and descriptive comments; a listing of achievements; marriage and dates; offspring names, date of birth, date of death; copies of birth certificates; copies of death certificates; reference to DNA box samples; and photos.

As described above, the genealogical organization system 26 provides an important improvement on known DNA collection and storage systems. The family tree 100 of the genealogical organization system 26 provides a quick view of the genealogy of the family and identifies known carries of genetic diseases as well as the location of their DNA samples in the kit 20. The genealogical organization system 26 also includes more detailed information on the individual in their information packet 104. It should be understood that the genealogical organization system 26 may also be generated electronically and stored on a disk, or CD-Rom stored within the case. The electronic genealogical organization system 26 may be used separately or in addition to the hard copy stored within the storage case 30.

Use of the Kit

Use of the kit 20 to collect and preserve vital data about and physical specimens from an individual is simple and easily carried out in the home. First, the individual is assigned a block 102 on the family tree corresponding to their position in the family and basic information such as the individual's name and date of birth is placed in the block 102. The individual is then given a number that is placed in their block 102. The number preferably identifies the DNA sample storage box 41 containing their samples as well as the individual's information packet 104. A photograph of the individual is then secured and attached to the corresponding information packet 104. All of the information solicited on the information packet 104 is then entered in the spaces provided on the packet. The packet may be updated as new events occur.

Next, the individual samples for the kit are then taken and stored. One or all of the described samples may be taken and stored in the storage box 41. The fingerprints may be taken on the identification card 49 and placed in the sleeve 51. Each finger of the person being identified is rolled across the inked pad 47 to deposit the fingerprint ink onto the fingertips. The fingers are then rolled in sequence across the identification card 49 in the positions of the matrix corresponding to each finger. Prints of the four fingers of the left and right hands are taken simultaneously and applied to their proper positions within the fingerprint matrix. The identification card 49 is then labeled, stored in the sleeve 51 and placed in the storage box 41.

Next, a sample of the individual's hair may be taken and prepared for preservation. Preferably, several hairs are cut from the individual's head or removed via a comb and secured to the back of the hair identification card 43 with a staple or tape. The sample is then set aside to air dry for approximately one hour such that oil and other fluids can evaporate from the sample. This drying time is important because fluids contained within freshly cut hair samples can accelerate the deterioration of the samples if they are sealed along with the samples. Once the hair sample has dried for the specified time, the identification of the individual from whom the sample was taken and the date it was taken are written on the identification card 43 and the card and its attached sample are secured and sealed within the plastic hair sleeve 71 and placed in the storage box 41.

Next a sample of the scrapings from the inner cheek may be taken using swabs 63. Once the swabs 63 have dried, the identification of the individual from whom the sample was taken and the date it was taken are provided on the identification card 69 and the card and its attached sample are secured and sealed within the plastic sleeve 65 and placed in the storage box 41.

A sample of the individual's blood may also be collected and prepared for preservation. In this regard, the cloth patch 55, lancing tool 53, and sterile swab 57 are obtained and the end of the lancing tool 53 is twisted off to reveal its blade. A fingertip of the individual being identified is then wiped with the swab and pierced quickly with the lancing tool 53 to draw a small amount of blood from the finger. The patch 55 is applied to the fingertip to absorb the blood for preservation. The blood impregnated patch 55 is then set aside for approximately one hour to allow unwanted fluids to evaporate from the blood sample. As with the hair sample, these fluids can accelerate the deterioration of the blood if sealed therewith such that the drying process is important for maximum longevity of the sample. The identification of the individual whose blood sample has been taken and the date the sample was taken are then written on the card 69 and the card and blood impregnated patch 55 are secured and sealed within the plastic sleeve 65 and placed in the storage box 41.

Once the samples are dried and sealed within their sleeves, one of the adhesive decals can be secured over the top edge of each sleeve to insure that the contents of the sleeves are not subsequently tampered with or altered. Once all of the samples have been collected the storage boxes 41 are placed in the case 30 for storage. It should be recognized that the storage boxes 41 may be prelabeled with an ID number or identifying symbol that will be placed in the block 102 on the family tree. Alternatively, one may label the box 41 after insertion of the samples.

In addition to the described samples other samples can be collected and stored such as skin scrapings, nasal wash or scrapings or secretions, eye secretions, mucous membranes, saliva, expectorant, toenails, fingernails, sputum, urine, urine sediment, genital secretions, sperm, semen, vaginal and cervical secretions, feces, fecal matter or other body sites.

The entire storage case 30 may be placed within the family freezer for storage however, such storage is not necessary. In the freezer, the blood and hair samples are chilled to a temperature below their freezing point such that the useful life of these samples is extended long beyond that which they would exhibit under normal temperature conditions. Further, the low temperature within the freezer tends to enhance the longevity of the fingerprints and slows the aging and deterioration of photographs and other identifying information.

The invention has been described above in terms of a preferred embodiment. It will be obvious, however, that many variations of the illustrated embodiment might well be contemplated by ordinarily skilled artisans. The order in which information and samples are taken and sealed can, for example, be different than that illustrated above. Further, various means for sealing the samples could also be used with results comparable to that of the plastic sleeves of the preferred embodiment.

Although the best mode contemplated by the inventors of carrying out the present invention is disclosed above, practice of the present invention is not limited thereto. It will be manifest that various additions, modifications and rearrangements of the features of the present invention may be made without deviating from the spirit and scope of the underlying inventive concept.

Moreover, as noted throughout the application the individual components need not be formed in the disclosed shapes, or assembled in the disclosed configuration, but could be provided in virtually any shape, and assembled in virtually any configuration, so as to provide for a DNA storage and family tree kit. Furthermore, all the disclosed features of each disclosed embodiment can be combined with, or substituted for, the disclosed features of every other disclosed embodiment except where such features are mutually exclusive.

It is intended that the appended claims cover all such additions, modifications and rearrangements. Expedient embodiments of the present invention are differentiated by the appended claims.

What is claimed is:

1. A method for collecting, storing and categorizing DNA and other genetic material across generations of a family comprising:
(a) collecting at least one sample of DNA from a family member;
(b) storing the sample in a DNA storage box;
(c) placing the box in a storage case; and
(d) recording information about the family member in a genealogical organization system that includes a family tree having indicia indicating the location of the family member's DNA storage box in the storage case.

2. The method of claim 1, wherein the step of collecting DNA comprises collecting a blood sample.

3. The method of claim 2, wherein the step of collecting a blood sample comprises:
(a) placing drops of blood on a card labeled for identification;
(b) drying the blood; and
(c) placing the card in a plastic sleeve.

4. The method of claim 1, wherein the step of collecting DNA comprises collecting cheek cells.

5. The method of claim 4, wherein the step of collecting cheek cells comprises:
(a) brushing the inside of the mouth cheek to collect cells with a swab;
(b) drying the swab; and
(c) placing the swab in a sleeve with a card labeled for identification.

6. The method of claim 1, wherein the step of collecting DNA comprises collecting a hair follicle.

7. The method of claim 6, wherein the step of collecting a hair follicle comprises:
(a) collecting loose head hair which accumulates in a comb;
(b) cutting off and discarding the end of the hair not containing hair follicles; and
(c) placing the hair strands containing hair follicles in sleeve with a card labeled for identification.

8. The method of claim 1, further comprising the step of taking fingerprints of the family member and placing the fingerprints in the DNA storage box.

9. The method of claim 1, wherein the step of collecting DNA comprises collecting multiple samples of DNA from blood, mouth cheek cells and hair follicles.

10. The method of claim 1, wherein the step of recording information about the family member in a genealogical organization system includes the step of filling out an information packet on an individual.

11. The method of claim 10, wherein the family tree includes indicia indicating the location of the information packet in the storage case.

12. The method of claim 10, wherein the information packet includes information on at least one of an individual's name, date of birth, date of death, sex, mothers maiden name, country of origin, eye color, hair color, vision, blood type, height, weight, diseases and age of incidents, injuries and age of incidents and prescriptions taken.

13. The method of claim 1, wherein the family tree includes indicia indicating a person is a known carrier of a genetic disease.

14. The method of claim 1, wherein the family tree includes indicia indicating a person's response to medication.

15. A DNA family tree kit comprising:
(a) a storage case configured to receive a plurality of DNA storage boxes associated with a corresponding group of family members;
(b) a means for collecting at least one sample of DNA from each family member; and
(c) a genealogical organization system that includes a family tree.

16. The DNA family tree kit of claim 15, wherein the family tree includes indicia indicating the location of a family member's DNA storage box within the storage case.

17. The DNA family tree kit of claim 15, further comprising an information packet in the storage case.

18. The DNA family tree kit of claim 15, wherein the family tree includes indicia indicating if a family member is a known carrier of a genetic disease.

19. DNA family tree kit of claim 15, wherein the family tree includes indicia indicating a family member's response to medication.

20. A method of assimilating and preserving genetic information about a family over multiple generations said method comprising the steps of:
(a) collecting data about the family members;
(b) collecting at least one sample containing DNA from each of the family members;
(c) sealing the collected samples against exposure to ambiance;
(d) storing each family member's DNA sample in a separate DNA storage box;
(e) placing the DNA storage boxes in a storage case; and
(e) recording the data about the family member in a genealogical organization system that includes a family tree having indicia indicating if a family member is a known carrier of a genetic disease.
